(19) United States
(12) Patent Application Publication
(10) Pub. No.: US 2004/0197264 A1
(43) Pub. Date: Oct. 7, 2004

(54) Microspheres comprising therapeutic and diagnostic radioactive isotopes

(76) Inventors: Alexander Schwarz, Brookline, MA (US); James A. Krom, Belmont, MA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/407,144

(22) Filed: Apr. 4, 2003

(51) Int. Cl. A61K 51/00

(52) U.S. Cl. 424/1.11

(57) ABSTRACT

One aspect of the present invention relates to a microsphere impregnated with a radioisotope that emits therapeutic β-particles and a radioisotope that emits diagnostic γ-radiation; wherein the atomic number of the first radioisotope is not the same as the atomic number of the second radioisotope. In one preferred embodiment, the microsphere is composed of glass impregnated with ⁹⁰Y as the source of the therapeutic β-emissions and ¹⁹⁸Au as the source of the diagnostic γ-emissions. Another aspect of the present invention relates to the preparation of a microsphere impregnated with a radioisotope that emits therapeutic β-particles and a radioisotope that emits diagnostic γ-radiation; wherein the atomic number of the first radioisotope is not the same as the atomic number of the second radioisotope. In one preferred embodiment, a glass microsphere containing ⁹⁰Y and ¹⁹⁸Au is prepared by neutron activation of a glass microsphere comprising glass, ⁸⁹Y and ¹⁹⁷Au. Another aspect of the present invention relates to administration to a mammal of a therapeutically effective amount of microspheres impregnated with a β-emitting radioisotope and a γ-emitting radioistope; wherein the atomic number of the first radioisotope is not the same as the atomic number of the second radioisotope. In one preferred embodiment, said microspheres are administered to the patient through a catheter.

### BACKGROUND OF THE INVENTION

The development of new and more effective treatments for cancer is of utmost concern. This is particularly relevant for the treatment of malignant tumors found in the liver owing to the current unsatisfactory treatment options. At the present time, the preferred method of treatment for patients with liver metastases is surgical resection. Unfortunately, the 5-year survival rate for patients that have undergone this form of treatment is only around 35%. Scheele J and Altendorf-Hofmann A. Resection of colorectal liver metastases. Langenbeck's Arch. Surg. 1999; 313-327. This disappointingly low survival rate is compounded by the fact that most tumours are inoperable by the time of diagnosis. Other treatment options for these tumours include conventional chemotherapy and external radiotherapy. Häfeli U O, Casillas S, Dietz D W, Pauer G J, Rybicki L A, Conzone S D and Day D E. Hepatic tumor radioembolization in a rat model

using radioactive rhenium (¹⁸⁶Re/¹⁸⁸Re) glass microspheres. Int. J. Radiation Oncology Biol. Phys. 1999; 44:189-199 and Link K H, Komnman M., Formentini A, Leder G, Sunelaitis E, Schatz M, Prelmar J and Beger H G. Regional chemotherapy of non-resectable liver metastases from colorectal cancer—literature and institutional review. Langenbeck's Arch. Surg. 1999; 384:344-353. Unfortunately, neither of the latter regimens have shown significant improvements in patient survival.

Recent developments in selective radionuclide therapy indicate that radiolabeled microspheres may offer a promising treatment option for patients suffering from a variety of types of cancer. This treatment allows the selective delivery of therapeutic radioactive particles to the tumor with as little surrounding tissue damage as possible. This new treatment option is particularly important for cancers with an extremely poor prognosis and without other adequate therapies, such as primary and metastatic malignancies of the liver. For example, the regional administration of therapeutic agents via the hepatic artery is one strategy that has been developed to improve tumour response. Bastian P, Bartkowski R, Kohler H and Kissel T. Chemo-embolization of experimental liver metastases. Part 1: distribution of biodegradable microspheres of different sizes in an animal model for the locoregional therapy. Eur. J. Pharm. Biopharm. 1998; 46:243-254. This form of treatment promises to be particularly effective for both primary and metastatic liver cancer since these tumors are well vascularized and receive the bulk of their blood supply from the hepatic artery. Ackerman N B, Lien W M, Kondi E S and Silverman N A. The blood supply of experimental liver metastases. The distribution of hepatic artery and portal vein blood to “small” and “large” tumors. Surgery 1969; 66:1067-1072. In addition, many kinds of radiolabeled particles and radionuclides have been tested for local treatment of a variety of tumors in organs, including liver, lung, tongue, spleen and soft tissue of extremities.

In early applications of this technique, yttrium oxide powder was suspended in a viscous medium prior to administration. Yttrium oxide was selected for the technique because it emits nearly 100 percent beta radiation. See Nolan et al., Intravascular Particulate Radioisotope Therapy, The American Surgeon 1969; 35: 181-188 and Grady et al., Intra-Arterial Radioisotopes to Treat Cancer, American Surgeon 1960; 26:678-684. However, the yttrium oxide powder had a high density (5.01 gm/cm ³) and irregular particle shape. The high density of pure yttrium oxide powder made it difficult to keep the particles in suspension in the liquids used to inject them into the body, and the sharp corners and edges of yttrium oxide particles also irritate surrounding tissue in localized areas. In later applications, the particles used have been microspheres composed of an ion exchange resin, or crystalline ceramic core, coated with a radioactive isotope such as P-32 or Y-90. Both ion exchange resin and crystalline ceramic microspheres offer the advantage of having a density much lower than that of yttrium oxide particles, and the ion exchange resin offers the additional advantage of being particularly easy to label. See Zielinski and Kasprzyk, Synthesis and Quality Control Testing of ³²P labelled Ion Exchange Resin Microspheres for Radiation Therapy of Hepatic Neoplasms, Int. J. Appl. Radiat. Isot. 1983; 34:1343-1350. In still another application, microspheres have been prepared comprising a ceramic material and having a radioactive isotope incorporated into the ceramic material. While the release of radioactive isotopes from a radioactive coating into other parts of the human body may be eliminated by incorporating the radioisotopes into ceramic spheres, the latter product form is nevertheless not without its disadvantages. Processing of these ceramic microspheres is complicated because potentially volatile radioactivity must be added to ceramic melts and the microspheres must be produced and sized while radioactive, with the concomitant hazards of exposure to personnel and danger of radioactive contamination of facilities.

The current technology often uses glass, resin, albumin, or polymer microspheres that are impregnated with a material that emits β-particles upon neutron activation. Research has indicated that the composition of the bead can be important in the design of an effective treatment. For example, glass is relatively resistant to radiation-damage, highly insoluble, and non-toxic. Glass can be easily spheridized in uniform sizes and has minimal radionuclidic impurities. Advances in manufacturing have led to the production of glass microspheres with practically no leaching of the radioactive material. Ho S, Lau W Y, Leung T W T, Chan M, Ngar Y K, Johnson P J and Li A K C. Clinical evaluation of the partition model for estimating radiation doses from yttrium-90 microspheres in the treatment of hepatic cancer. Eur. J. Nucl. Med. 1997; 24:293-298.

Although glass spheres have several advantages, their high density (3.29 g/ml) and non-biodegradability are major drawbacks. Mumper R J, Ryo U Y and Jay M. Neutron activated holmium-166-Poly(L-lactic acid) microspheres: A potential agent for the internal radiation therapy of hepatic tumours. J. Nucl. Med. 1991; 32:2139-2143 and Turner J H, Claringbold P G, Klemp P F B, Cameron P J, Martindale A A, Glancy R J, Norman P E, Hetherington E L, Najdovski L and Lambrecht R M. ¹⁶⁶Ho-microsphere liver radiotherapy: a preclinical SPECT dosimetry study in the pig. Nucl. Med. Comm. 1994; 15:545-553. The relatively high density increases the chance of intravascular settling. Ho S, Lau W Y, Leung T W T and Johnson P J. Internal radiation therapy for patients with primary or metastatic hepatic cancer. Cancer 1998; 83:1894-1907. Nevertheless, glass microspheres produced under the name TheraSpheres® were the first registered microsphere product for internal radionuclide therapy, and have been used in patients with primary or metastatic tumours. In comparison, only a few radioisotopes have the characteristics necessary for the treatment of tumors. Important characteristics of a suitable radioisotope would include a radiational spectrum (strength of β-particle emision) appropriate to the size of the tumor, high dose rate, short half-life, and γ-emission for external imaging.

The most suitable radioactive materials are yttrium-90, rhenium-188 and holmium-166. All three of these materials emit β-radiation useful for radiotherapy. Although ⁹⁰Y is often used in radionuclide therapy, yttrium-90 has two major disadvantages for use in radiotherapy. First, long neutron activation times (>2 weeks) are needed to achieve therapeutic activities of yttrium because ⁹⁰Y's precursor has a small thermal neutron cross section of 1.28 barn. Secondly, the biodistribution of microspheres loaded with ⁹⁰Y cannot be directly determined in clinical trials, since ⁹⁰Y is a pure β-emitter and does not produce imageable γ-rays. Natural rhenium is composed of two isotopes, ¹⁸⁵Re and ¹⁸⁷Re, that form β-emitting ¹⁸⁶Re and ¹⁸⁸Re radioisotopes, respectively, upon neutron activation. The nuclear and dosimetric properties of the rhenium radioisotopes are comparable to those of ⁹⁰Y, but they have imageable γ-photons. Like the rhenium radioisotopes, ¹⁶⁶Ho emits β-particles and photons and has a relatively short physical half-life of 26.8 h, compared to ⁹⁰Y (64.1 h) and ¹⁸⁶Re (90.6 h), resulting in a high dose rate.

The development of microspheres for radionuclide therapy is complicated by the difficulty in determining the biodistribution of the microspheres in vivo, as noted above for ⁹⁰Y. The biodistribution of microspheres is critically important for this type of radiotherapy because the microsphere must be in close proximity to the tumor being treated. One potential solution to this problem would be to attach a material to the microsphere that emits a detectable, non-hazardous signal.

### SUMMARY OF THE INVENTION

One aspect of the present invention relates to a microsphere, comprising a material selected from the group consisting of glass, polymer and resin; a first radioisotope that emits a therapeutic β-particle; and a second radioisotope that emits a diagnostic γ-ray; wherein the atomic number of the first radioisotope is not the same as the atomic number of the second radioisotope.

The present invention also relates to a method of preparing a radioactive microsphere, comprising the steps of: combining a non-radioactive precursor of a first radioisotope, a non-radioactive precursor of a second radioisotope, and a material selected from the group consisting of glass, polymer, and resin, to form a mixture; wherein the atomic number of the first radioisotope is not the same as the atomic number of the second radioisotope; fabricating a microsphere from said mixture; and bombarding said microsphere with neutrons.

Another aspect of the present invention relates to a method of treating a mammal suffering from a medical condition, comprising the step of administering to said mammal a therapeutically effective amount of radioactive microspheres each comprising a material selected from the group consisting of glass, polymer, and resin; a first radioisotope that emits a therapeutic β-particle; and a second radioisotope that emits a diagnostic γ-ray; wherein the atomic number of the first radioisotope is not the same as the atomic number of the second radioisotope.

### DETAILED DESCRIPTION OF THE INVENTION

The invention will now be described more fully with reference to the accompanying examples, in which certain preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

### Overview of a Preferred Embodiment

Radionuclide therapeutic techniques using microspheres for the treatment of various cancers rely upon the precise and accurate delivery of microspheres to a tumor. This treatment option offers the promise of delivering therapy directly to the tumor cells which minimizes damage to nearby healthy tissue, a serious shortcoming associated with conventional treatment options such as chemotherapy, radiotherapy, or surgical resection. However, the effectiveness of cancer treatments using radionuclide microspheres is often hampered by the inability to determine the biodistribution of the microspheres. Therefore, a non-invasive method to determine the biodistribution of said microspheres would be highly useful. Microspheres containing ¹⁹⁸Au, for emission of γ-radiation to enable detection, have been designed that incorporate ⁹⁰Y for emission of β-particles useful in the treatment of various medical conditions. A method to determine the amount of ¹⁹⁷Au required per microsphere has been established based on the physical properties and relative proportions of ⁹⁰Y, ¹⁹⁸Au, and the bulk material comprising the bead. A mathematical formula has been derived that allows for the calculation of the necessary quantity of the stable isotope of the therapeutic β-emitting radionuclide and ¹⁹⁷Au. The composition and size of the microsphere may be customized to best fit a particular application. The radiolabeled microspheres may be introduced into a subject mammal in accord with standard procedures and the biodistribution of the microspheres may be determined by detection of the gamma-rays emitted by ¹⁹⁸Au.

### Derivation of the Rate of Radioactive Decay

Neutron activation of a stable isotope to give a radioactive isotope is described by the simple scheme where k_{N} and k_{D} are the neutron capture constant and radioactive decay constant, respectively.

$$A {⟶}^{{k}_{N}} {A}^{*} {⟶}^{{k}_{D}} P$$

A, A*, and P represent the numbers of atoms (or moles) of the stable isotope, radioactive isotope, and decay product, respectively. The net rate of formation of the radioactive isotope A* is given by

$$\frac{ⅆ {A}^{*}}{ⅆ t} = {k}_{N} A - {k}_{D} {A}^{*} \qquad ( Eq 1 )$$

which has the solution, assuming no A* is present initially,

$${A}^{*} = \frac{{k}_{N} {A}_{0}}{{k}_{D} - {k}_{N}} ( {ⅇ}^{- {k}_{N} t} - {ⅇ}^{- {k}_{D} t} ) \qquad ( Eq 2 )$$

where A₀ is the initial quantity of the stable isotope.

The radioactive decay constant can be expressed in terms of the radioactive isotope's half-life (t_{1/2}) according to:

$${k}_{D} = \frac{ln ( 2 )}{{t}_{1 / 2}} \qquad ( Eq 3 )$$

The neutron capture constant is determined by the neutron flux φ and the neutron capture cross-section χ according to: Constants φ and χ are typically expressed in units of cm ⁻²s⁻¹ and 10⁻²⁴cm² (barns), respectively.

After removing the material from the neutron source, the radioactive isotope will decay at a rate given by

$$\frac{ⅆ {A}^{*}}{ⅆ t} = - {k}_{D} {A}^{*} \qquad ( Eq 5 )$$

### Determination of Required Quantity of Stable Isotope

The radioactivity, defined by -dA*/dt, is usually expressed in units of s⁻¹ (becquerel) or 3.7×10¹⁰ s⁻¹ (curie). From Eq 2 and Eq 5, the equation that expresses the radioactivity at the time of removal of the sample from the neutron source is

$$\frac{- ⅆ {A}^{*}}{ⅆ t} = \frac{{k}_{D} {k}_{N} {A}_{0} ( {ⅇ}^{- {k}_{N} t} - {ⅇ}^{- {k}_{D} t} )}{{k}_{D} - {k}_{N}} \qquad ( Eq 6 )$$

Solving Eq 6 for A₀ allows calculation of the quantity of stable isotope that is required to achieve a desired radioactivity after an irradiation time t according to:

$${A}_{0} = \frac{ⅆ {A}^{*} {k}_{D} - {k}_{N}}{ⅆ t {k}_{D} {k}_{N} ( {ⅇ}^{- {k}_{N} t} - {ⅇ}^{- {k}_{D} t} )} \qquad ( Eq 7 )$$

### Identity of β-Emitting Therapeutic Radionuclide

A radionuclide suitable for internal radionuclide therapy of primary and metastatic malignancies must have the following properties: First, the radioisotope must have an appropriate radiation spectrum for treating small to large multiple tumours. Large tumours with a vascular periphery but a necrotic centre take up less microspheres per volume; therefore, a high energy β-emitter with a subsequently high tissue range is needed to reach the interior of the tumour. Second, a high dose rate is advantageous for the radiobiological effect. Spencer R P. Applied principles of radiopharmaceutical use in therapy. Nucl. Med. Biol. 1986; 13:461-463 and Spencer R P. Short-lived radionuclides in therapy. Nucl. Med. Biol. 1987; 14:537-538. Consequently, a short half-life is preferable. Third, a γ-emitter is desirable for external imaging to determine the biodistribution of the radioisotope with a gamma camera. However, the radioactivity should be low to prevent unnecessary radiation burden to the patient and environment. Mumper R J, Ryo U Y and Jay M. Neutron activated holmium-166-Poly(L-lactic acid) microspheres: A potential agent for the internal radiation therapy of hepatic tumours. J. Nucl. Med. 1991; 32:2139-2143. Further, the labeling of particles has to be simple without any leakage of the isotope. Finally, a large thermal neutron cross section is needed to enable high specific activities to be achieved within short neutron activation times. Conzone S D, Häfeli U O, Day D E and Ehrhardt G J. Preparation and properties of radioactive rhenium glass microspheres intended for in vivo radioembolization therapy. J. Biomed. Mater. Res. 1998; 42:617-625. Unfortunately, only a few radioisotopes have characteristics which make them potentially suitable for the treatment of tumours. Suitable radionuclides are selected from the group consisting of ⁹⁰Y, ^{99m}Tc, ¹⁸⁸Re, ³²P, ¹⁶⁶Ho, ¹⁰⁹Pd, ¹⁴⁰La, ¹⁵³Sm, ¹⁶⁵Dy, and ¹⁶⁹Er. In preferred embodiments, the radionuclide is ⁹⁰Y, ¹⁶⁶Ho, or ¹⁸⁸Re.

### Detection of γ Photons Emitted by Diagnostic Radionuclide

Today, cancer is often found using a gamma camera, which provides images of potential tumors in the body by detecting the radiation emitted by a radiopharmaceutical given to a patient undergoing a full-body scan. In such systemic approaches, suspected tumor regions collect higher concentrations of the radiopharmaceutical, which produces a higher count rate and therefore a detectable contrast between the tumor region and its surroundings.

Most clinically-used radiopharmaceuticals are diagnostic agents incorporating a gamma-emitting nuclide which, because of physical or metabolic properties of its coordinated ligands, localizes in a specific organ after intravenous injection. The resultant images can reflect organ structure or function. These images are obtained by means of a gamma camera that detects the distribution of ionizing radiation emitted by the radioactive molecules. The principal isotope currently used in clinical diagnostic nuclear medicine is metastable technetium-99m, which has a half-life of 6 hours.

As outlined above, a gamma camera is used in nuclear medicine for the display, in an organ, of the distribution of molecules marked by a radioactive isotope injected into a patient. Thus, a gamma camera has a collimator to focus the gamma photons emitted by the patient's body, a scintillator crystal to convert the gamma photons into light photons or scintillations, and an array of photomultiplier tubes, each of which converts the scintillations into electrical pulses. A detection system such as this is followed by a processing and display unit that can be used to obtain an image projection of the distribution of the radioactive isotopes in the patient during the acquisition of the image.

### Activation of Radionuclide

A variety of neutron sources, such as reactors, accelerators, and radioisotopic neutron emitters, can be used for radioactivation of stable isotopes by neutron activation. Systems and methods for neutron activation are described in U.S. Pat. Nos. 6,149,889 and 6,328,700. Nuclear reactors with their high fluxes of neutrons from uranium fission offer the highest available activation rates for most elements. Different types of reactors and different positions within a reactor vary considerably with regard to their neutron energy distributions and fluxes due to the materials used to moderate (or reduce the energies of) the primary fission neutrons. However, most neutron energy distributions are quite broad and include three principal components (thermal, epithermal, and fast).

The thermal neutron component includes low-energy neutrons (energies below 0.5 eV) in thermal equilibrium with atoms in the reactor's moderator. At room temperature, the energy spectrum of thermal neutrons is best described by a Maxwell-Boltzmann distribution with a mean energy of 0.025 eV and a most probable velocity of 2200 m/s. In most reactor irradiation positions, 90-95% of the neutrons that bombard a sample are thermal neutrons.

The epithermal neutron component includes neutrons (energies from 0.5 eV to about 0.5 MeV) which have been only partially moderated. A cadmium foil 1 mm thick absorbs all thermal neutrons, but will allow epithermal and fast neutrons above 0.5 eV in energy to pass through. In a typical unshielded reactor irradiation position, the epithermal neutron flux represents about 2% the total neutron flux. Both thermal and epithermal neutrons induce reactions on target nuclei.

The fast neutron component of the neutron spectrum (energies above 0.5 MeV) includes the primary fission neutrons which still have much of their original energy following fission. Fast neutrons contribute very little to the reaction, but instead induce nuclear reactions where the ejection of one or more nuclear particles—(n,p), (n,n′), and (n,2n)—are prevalent. In a typical reactor irradiation position, about 5% of the total flux consists of fast neutrons.

The amount of radiation delivered to a target by radioactive metal or metal compound labeled microspheres can be controlled in a variety of ways; for example, by varying the amount of metal associated with the spheres, the extent of radioactivation of the metal, the quantity of microspheres administered, and the size of microspheres administered.

### Bulk Composition of Microspheres

Glass

Glass is relatively resistant to radiation-damage, highly insoluble, and non-toxic. Glass can be easily spheridized in uniform sizes and has minimal radionuclidic impurities. Advances in technology have led to the production of glass microspheres with practically no leaching of radioactive material. Ho S, Lau W Y, Leung T W T, Chan M, Ngar Y K, Johnson P J and Li A K C. Clinical evaluation of the partition model for estimating radiation doses from yttrium-90 microspheres in the treatment of hepatic cancer. Eur. J Nucl. Med. 1997; 24:293-298. Although the glass spheres have several advantages, their high density (3.29 g/ml) and their non-biodegradability are drawbacks. Mumper R J, Ryo U Y and Jay M. Neutron activated holmium-166-Poly(L-lactic acid) microspheres: A potential agent for the internal radiation therapy of hepatic tumours. J. Nucl. Med. 1991; 32:2139-2143. The relatively high density of glass increases the chance of intravascular settling. Glass microspheres produced under the name TheraSpheres® were the first registered microsphere product for internal radionuclide therapy, and are used in patients with primary or metastatic tumours. Because of the lack of γ-emission of ⁹⁰Y, radioactive rhenium (¹⁸⁶Re/¹⁸⁸Re) microspheres were also produced. The general method of manufacture of these spheres was the same as for the ⁹⁰Y spheres. Häfeli U O, Casillas S, Dietz D W, Pauer G J, Rybicki L A, Conzone S D and Day D E. Hepatic tumor radioembolization in a rat model using radioactive rhenium (¹⁸⁶Re/¹⁸⁸Re) glass microspheres. Int. J. Radiation Oncology Biol. Phys. 1999; 44:189-199 and Conzone S D, Häfeli U O, Day D E and Ehrhardt G J. Preparation and properties of radioactive rhenium glass microspheres intended for in vivo radioembolization therapy. J. Biomed. Mater. Res. 1998; 42:617-625.

Brown et al. prepared ¹⁶⁶Ho-loaded glass particles for direct injection into tumours of mice, which resulted in an effective modality for deposition of intense γ-radiation for use in localized internal radionuclide therapy; however, no further studies were done. Brown R F, Lindesmith L C and Day D E. 166-Holmium-containing glass for internal radiotherapy of tumors. Int. J. Rad. Appl. Instrum. B 1991; 18:783-790.

Kawashita et al. suggested the use of phosphorus-rich Y₂O₃—Al₂O₃—SiO₂-glass microspheres containing phosphorus ions, which were produced by thermoelectron bombardment of red phosphorus vapour and implanted into glass, thus resulting in a high phosphorus content and high chemical durability. After activation by neutron bombardment the glass contains phosphorus-32 (³²P). Kawashita M, Miyaji F, Kokubo T, Takaoka G H, Yamada I, Suzuki Y and Inoue M. Surface structure and chemical durability of P⁺-implanted Y₂O₃—Al₂O₃—SiO₂ glass for radiotherapy of cancer. J. Non-Cryst. Solids 1999; 255:140-148.

Resins

Resin-based microspheres are favoured for radio-embolization. Chloride salts of holmium and yttrium have been added to cation exchange resins. Different resins were investigated by Schubiger et al., amongst which were Bio-Rex 70, Cellex-P, Chelex 100, Sephadex S P and A G 50W-X8. Schubiger P A, Beer H-F, Geiger L, Rösler H, Zimmerman A, Triller J, Mettler D and Schilt, W. ⁹⁰Y-resin particles-animal experiments on pigs with regard to the introduction of superselective embolization therapy. Nucl. Med. Biol. 1991; 18:305-311. The resins with ⁹⁰Y bound to the carboxylic acid groups of the acrylic polymer were sterilized and used for renal embolization of pigs. Only the pre-treated Bio-Rex 70 resulted in usable particles, with a retention of beta activity in the target organ of >95% of injected dose, and no histologically detectable particles in lung tissue samples. Zimmerman A, Schubiger P A, Mettler D, Geiger L, Triller J and Rösler H. Renal pathology after arterial yttrium-90 microsphere administration in pigs. A model for superselective radioembolization therapy. Invest. Rad. 1995; 30:716-723.

Aminex resins (Bio-Rad Inc. Hercules Calif., USA) loaded with ¹⁶⁶Ho or ¹⁸⁸Re also resulted in usable preparations. Turner et al. prepared microspheres by addition of ¹⁶⁶Ho-chloride to the cation exchange resin Aminex A-5, which has sulphonic acid functional groups attached to styrene divinylbenzene copolymer lattices. Turner J H, Claringbold P G, Klemp P F B, Cameron P J, Martindale A A, Glancy R J, Norman P E, Hetherington E L, Najdovski L and Lambrecht R M. ¹⁶⁶Ho-microsphere liver radiotherapy: a preclinical SPECT dosimetry study in the pig. Nucl. Med. Comm. 1994; 15:545-553. Reproducible, non-uniform distributions of the ¹⁶⁶Ho-microspheres throughout the liver were observed on scintigraphic images, following intrahepatic arterial administration in pigs. This predictable distribution allowed these investigators to determine the radiation absorbed dose from a tracer activity of ¹⁶⁶Ho-microspheres, and to define the administered activity required to provide a therapeutic dose. Aminex A-27 was labelled with ¹⁸⁸Re by adding ¹⁸⁸Re-perrhenate and SnCl₂ to vacuum-dried resin particles. Wang S-J, Lin W-Y, Chen M.-N, Chi C-S, Chen J-T, Ho W-L, Hsieh B-T, Shen L-H, Tsai Z-T, Ting G, Mirzadeh S and Knapp F F. Intratumoral injection of rhenium-188microspheres into an animal model of hepatoma. J Nucl. Med. 1998; 39:1752-1757. The mixture was boiled and centrifuged and microspheres were separated and resuspended in saline. Spheres were tested by direct intratumoural injection into rats with hepatoma. Survival over 60 days was significantly better in the treated versus the control group (80% vs. 27%).

Investigators from Australia and Hong Kong have used unspecified resin-based particles labeled with ⁹⁰Y for treatment of patients with primary or secondary liver cancer. Lau W Y, Leung W T, Ho S, Leung N W Y, Chan M, Lin J, Metreweli C, Johnson P and Li A K C. Treatment of inoperable hepatocellular carcinoma with intrahepatic arterial yttrium-90 microspheres: a phase I and II study. Br. J. Cancer 1994; 70:994-999. The spheres had a diameter of 29-35 μm, a density of 1.6 g/mL and a specific activity of approximately 30-50 Bq per sphere. Treatment was well tolerated with no bone-marrow or pulmonary toxicity. The median survival was 9.4 months (range 1.8-46.4) in 71 patients, and the objective response rate in terms of drop in tumour marker levels was higher than that based on reduction in tumour volume shown by computed tomography. Lau W Y, Ho S, Leung T W T, Chan M, Ho R, Johnson P J and Li A K C. Selective internal radiation therapy for nonresectable hepatocellular carcinoma with intraarterial infusion of ⁹⁰yttrium microspheres. Int. J. Radiation Oncology Biol. Phys. 1998; 40:583592.

Albumin

Since 1969, technetium-99m-microspheres (^{99m}Tc-microspheres) of human serum albumin (HSA) have been widely used for clinical nuclear medicine, particularly for lung scanning. Wunderlich G, Pinkert J, Andreeff M, Stintz M, Knapp F F, Kropp J and Franke W G. Preparation and biodistribution of rhenium-188 labeled albumin microspheres B 20: a promising new agent for radiotherapy. Appl. Radiat. Isotopes 2000; 52:63-68 and Rhodes B A, Zölle I, Buchanan J W and Wagner H N. Radioactive albumin microspheres for studies of the pulmonary circulation. Radiology 1969; 92:1453-1460. ¹⁸⁸Re-labeled HSA microspheres used by Wunderlich et al. are uniform in size, with a mean diameter of 25 μm, and are biocompatible and biodegradable. However, the labeling process is time-consuming and depends on SnCl₂2H₂O and gentisic acid concentration. On the surface of the microspheres a shell of less than about 1 μm thickness was seen, probably consisting of precipitated tin hydroxide. The particle labeling (coating) may be achieved by a combination of the reduction reaction of RE(VII) with Sn(II) and a particle surface-related coprecipitation effect of tin hydroxide colloid with high adsorption capacity and reduced, hydrolysed rhenium. The labeling yield under optimal reaction conditions is more than 70%. Biodistribution experiments in rats, using the lungs as a model for a well-perfused tumour, resulted in excellent in vivo stability.

As well as rhenium, yttrium has been bound to HSA for internal radiotherapy. Watanabe N, Oriuchi N, Endo K, Inoue T, Tanada S, Murata H and Sasaki Y. Yttrium-90 labeled human macroaggregated albumin for internal radiotherapy: combined use with DTPA. Nucl. Med. Biol. 1999; 26:847-851. ⁹⁰Y-acetate and macroaggregates of HSA (MAA) (Macrokit®, Dainabot, Tokyo, Japan) were suspended in sodium acetate buffer and incubated at room temperature. Experiments in mice were carried out in order to investigate the possibility of using ⁹⁰Y-MAA as an internal radiotherapeutic agent for whole-lung irradiation. Yttrium-activity in the lung was cleared within 72 h post injection and activity was redistributed in other organs, especially in the bone, but this could be prevented by the combined use of CaNa₃DTPA. Based on its rapid clearance ⁹⁰Y-MAA was suggested as being useful for fractionated internal radiotherapy of the lung.

Polymers

Polymer-based microspheres have many advantages over other materials, in particular their near-plasma density, biodegradability and biocompatibility. However, the major disadvantage is their inability to withstand high thermal neutron fluxes. Conzone S D, Häfeli U O, Day D E and Ehrhardt G J. Preparation and properties of radioactive rhenium glass microspheres intended for in vivo radioembolization therapy. J. Biomed. Mater. Res. 1998; 42:617-625. Additives and adjustment of irradiation-parameters can overcome this problem. A solvent evaporation technique has been used for preparation of poly(L-lactic acid) (PLLA) microspheres containing ¹⁶⁶Ho, ⁹⁰Y and ¹⁶Re/⁸⁸Re. Mumper et al. has prepared PLLA microspheres with holmium-165-acetylacetonate (HoAcAc). Mumper R J and Jay M. Poly(L-lactic acid) microspheres containing neutron-activatable holmium-165: A study of the physical characteristics of microspheres before and after irradiation in a nuclear reactor. Pharm. Res. 1992; 9:149-154. HoAcAc complex and PLLA were dissolved in chloroform and the solution was added to a polyvinyl alcohol (PVA) solution and stirred until the solvent had evaporated. Microspheres were graded and collected according to size, on stainless steel sieves having 20-50 μm openings. These microspheres can be dispensed in patient-ready doses that only need to be activated by neutron bombardment to a therapeutic amount of radioactivity in a nuclear reactor. These holmium loaded microspheres are currently being tested by intrahepatic arterial administration to rat liver tumours. A seven-fold increase of the ¹⁶⁶Ho microspheres in and around the tumour compared with normal liver was found, based on distribution of radioactivity.

Magnetic PLLA microspheres loaded with yttrium were made by Hafeli et al. in order to direct them to the tumour. Häfeli U O, Sweeney S M, Beresford B A, Humm J L and Macklis R M. Effective targeting of magnetic radioactive ⁹⁰Y-microspheres to tumor cells by an externally applied magnetic field. Preliminary in vitro and in vivo results. Nucl. Med. Biol. 1995; 22:147-155. This method resulted in stably loaded spheres, with the possibility of pre- or afterloading. To produce preloaded microspheres, PLLA was dissolved with L-α-phosphatidylcholine in methylene chloride. Commercially available ⁹⁰YCl₃ and magnetite Fe₃O₄ were added to the solution, vortexed, and sonicated. The suspension was injected into PBS with PVA, and microspheres were prepared following a solvent evaporation technique. Afterloaded spheres were prepared by suspending dried microspheres in a solution of PBS, after which ⁹⁰YCl₃ in HCl was added. Spheres were subsequently vortexed, incubated, and washed, resulting in labeled microspheres. Leaching of ⁹⁰Y was around 4% after 1 day in PBS at 37° C. Specific activity was 1.85 MBq/mg in both methods. ⁹⁰Y was bound to the carboxylic endgroups of the PLLA. Experiments in mice showed a 12-fold increase in activity in the tumour with a directional magnet fixed above it. Rhenium loaded PLLA microspheres were also developed, but these microspheres were unable to withstand the high neutron fluxes in a nuclear reactor which are necessary to achieve the high specific activity required in the treatment of liver tumours. Häfeli U O, Casillas S, Dietz D W, Pauer G J, Rybicki L A, Conzone S D and Day D E. Hepatic tumor radioembolization in a rat model using radioactive rhenium (¹⁸⁶Re/¹⁸⁸Re) glass microspheres. Int. J. Radiation Oncology Biol. Phys. 1999; 44:189-199.

### Manufacture of Microspheres

In certain cases, such as described in U.S. Pat. No. 5,302,369, microspheres have been prepared from a homogenous mixture of powders (i.e., the batch) that is melted to form the desired glass composition. The exact chemical compounds or raw materials used for the batch is not critical so long as they provide the necessary oxides in the correct proportion for the melt composition being prepared. For instance, if a YAS glass is being made, then yttria, alumina, and silica powders could be used as the batch raw materials. The purity of each raw material is preferably greater than 99.9%. After either dry or wet mixing of the powders to achieve a homogeneous mixture, the mixture may be placed in a platinum crucible for melting. High purity alumina crucibles can also be used if at least small amounts of alumina can be tolerated in the glass being made. The crucibles containing the powdered batch are then placed in an electric furnace which is heated 1500° to 1600° C., depending upon the composition. In this temperature range, the batch melts to form a liquid which is stirred several times to decrease its chemical heterogeneity. The melt should remain at 1500° to 1600° C. until all solid material in the batch is totally dissolved, usually 2-5 hours being sufficient. When melting and stirring is complete, the crucible is removed from the furnace and the melt is quickly quenched to a glass by pouring the melt onto a cold steel plate or into clean water. This procedure breaks the glass into fragments, which aids and simplifies crushing the glass to a fine powder. The powder is then sized and spheroidized for use.

Where it is desired to use microspheres having a diameter in the range of about 20 to about 30 micrometers, as for in the treatment of liver cancer, it is preferred that the quenched and broken glass be first crushed to about minus 100 mesh particles using a mortar and pestle. The minus 100 mesh material is then ground using a mechanized mortar and pestle or ball mill, until it passes a 400 mesh sieve. The particles are formed into glass microspheres by introducing the −400 mesh particles into a gas/oxygen flame where they are melted and a spherical liquid droplet is formed by surface tension. The droplets are rapidly cooled before they touch any solid object so that, their spherical shape is retained in the solid product.

Just prior to spheroidizing, the −400 mesh powder is rescreened through a 400 mesh sieve to remove any large agglomerates that may have formed during storage. The −400 mesh powder is then placed in a vibratory feeder located above the gas/oxygen burner. The powder is slowly vibrated into a vertical glass tube which guides the falling powder particles directly into the hot flame of a gas/oxygen burner. Any burner capable of melting −400 mesh particles of the particular glass composition being used is satisfactory. A typical rate for feeding the powder to the flame is 5 to 25 gm/hr with the described apparatus. The flame of the burner is directed into a metal container which catches the small glass beads as they are expelled from the flame. This container can be made of any metal which can withstand the heat of the burner and does not contaminate the glass. The container needs to be large enough so that the molten spheres can cool and become rigid before hitting a solid surface.

After spheroidization, the glass spheres are collected and rescreened. When the microspheres are intended to be used in the treatment of liver cancer, the fraction less than 30 and greater than 20 micrometers in diameter is recovered since this is the desirable size for use in the human liver. After screening, the −30/+20 microspheres are examined with an optical microscope and are then washed with a weakly acidic solution, filtered, and washed several times with reagent grade acetone. The washed spheres are then heated in a furnace in air to 500°-600° C. for 2-6 hours to destroy any organic material.

The final step is to examine a representative sample of the −30/+20 spheres in a scanning electron microscope to evaluate the size range and shape of the spheres. The quantity of undersize spheres (less than 10 micrometers in diameter) is determined along with the concentration of non-spherical particles. The composition of the spheres can be checked by energy dispersive x-ray analysis to confirm that the composition is correct and that there is an absence of chemical contamination. The glass microspheres are then ready for irradiation and subsequent administration to the patient.

Polymer-based microspheres used for internal radionuclide therapy are mainly prepared by a solvent evaporation technique. In the solvent evaporation process, the polymer is dissolved in a suitable water immiscible volatile solvent, and the medicament is dispersed or dissolved in this polymeric solution. The resulting solution or dispersion is then emulsified by stirring in an aqueous continuous phase, thereby forming discrete droplets. In order that the microspheres should form, the organic solvent must first diffuse into the aqueous phase and then evaporate at the water/air interface. As solvent evaporation occurs the microspheres harden, and free flowing microspheres can be obtained after suitable filtration and drying. O'Donnell P B and McGinity J W. Preparation of microspheres by solvent evaporation technique. Adv. Drug Del. Rev. 1997; 28:25-42

### Administration of Microspheres

The microspheres may be administered to the patient through the use of catheters either alone or in combination with vasoconstricting agents or by any other means of administration that effectively causes the microspheres to become embedded in the cancerous or tumor bearing tissue. See U.S. Pat. No. 5,302,369. For purposes of administration, the microspheres are preferably suspended in a medium that has a sufficient density or viscosity that prevents the microspheres from settling out of suspension during the administration procedure. Presently, preferred liquid vehicles for suspension of the microspheres include polyvinylpyrrolidone (PVP), sold under the trade designation Plasdone K-30 and Povidone by GAF Corp, a contrast media sold under the trade designation Metrizamide by Nyegard & Co. of Oslo, Norway, a contrast media sold under the trade designation Renografin 76 by E. R. Squibb & Co., 50% dextrose solutions and saline.

### Selected Clinical Applications of Radionuclide Microspheres

Given the increased skills of interventional radiologists, there is increasing interest in selective radionuclide therapy. Many kinds of radiolabeled particles and radionuclides have been tested for local treatment of a variety of tumours in organs, including liver, lung, tongue, spleen and soft tissue of extremities. The purpose of this treatment is the superselective application of suitable radioactive (high energetic β-emitters) particles to deliver high doses to the tumour, with as little surrounding tissue damage as possible. These new treatment methods are promising particularly for cancers with a poor prognosis and without other adequate therapies, such as primary and metastatic malignancies of the liver.

### Liver Cancer

Patients with primary or metastatic tumours were treated by radio-embolization via a catheter or direct injection of beads into the tumour with a needle. Gray B N, Burton M A, Kelleher D, Klemp P and Matz L. Tolerance of the liver to the effects of yttrium-90 radiation. Int. J. Radiation Oncology Biol. Phys. 1990; 18:619-623 and Tian J-H, Xu B-X, Zhang J-M, Dong B-W, Liang P and Wang X-D. Ultrasoundguided internal radiotherapy using yttrium-90-glass microspheres for liver malignancies. J. Nucl. Med. 1996; 37:958-963. Most studies describe administration of microspheres to patients via a catheter, whereby the tip was placed in the hepatic artery. The spheres eventually lodge in the microvasculature of the liver and tumour, remaining until the complete decay of the radioisotope. Lung shunting and tumour-to-normal liver ratio was determined after infusion of ^{99m}Tc-labeled macroaggregated albumin, and microspheres were subsequently administered to patients. Ho S, Lau W Y, Leung T W T, Chan M, Chan K W, Lee W Y, Johnson P J and Li A K C. Tumour-to-normal ratio of ⁹⁰Y microspheres in hepatic cancer assessed with ^{99m}Tc macroaggregated albumin. Brit. J. Rad. 1997; 70:823-828. Tumour-to-normal liver ratio was approximately 3-5. Yorke E D, Jackson A, Fox R A, Wessels B W and Gray N. Can current models explain the lack of liver complications in Y-90 microsphere therapy? Clin. Cancer Res. 1999; 5:3024s-3030s. In some studies the blood flow within the liver was temporarily redirected in favour of the tumour by a bolus infusion of a vasoconstrictor, and the spheres were then embolized into the arterial circulation. While external beam radiation causes radiation hepatitis at doses above 30-35 Gy the liver can tolerate up to 80-150 Gy, using internal radionuclide therapy. Ingold J, Reed G, Kaplan H and Bagshaw M. Radiation hepatitis. Am. J. Roentgenol. Radium Ther. Nucl. Med. 1965; 93:200-208. Increased longevity, pain relief, tumour response and total clinical improvement are frequently reported.

Head and Neck Cancers

Chemo-embolization with ethylcellulose microspheres of 100-450 μm has been used in the treatment of maxillary tumours. The role of intra-arterial radioisotope therapy in the treatment of head and neck cancer is just beginning in rabbits, in the work of van Es et al. Van Es R J J, Franssen O, Dullens H F J, Bemsen M R, Bosman F, Hennink W E and Slootweg P J. The VX2 carcinoma in the rabbit auricle as an experimental model for intra-arterial embolization of head neck squamous cell carcinoma with hydrogel dextran microspheres. Lab. Anim. 1999; 33:175-184. The optimal size of microspheres for treatment of unresectable head-and-neck cancer is still to be established. Some embolizations in the treatment of head-and-neck cancer have been carried out with particles of 100-450 pm. Tomura N, Kato K, Hirano H, Hirano Y and Watarai J. Chemoembolization of maxillary tumors via the superficial temporal artery using a coaxial catheter system. Radiation Med. 1998; 16:157.

Other Cancers

Intra-arterial administration of ⁹⁰Y-microspheres has been carried out in the spleen. Ariel I M and Padula G. Irradiation of the spleen by the intra-arterial administration of ⁹⁰yttrium microspheres in patients with malignant lymphoma. Cancer 1972; 31:90-96. Of nine patients with lymphosarcoma, five manifested no clinical response after splenic irradiation. One patient who complained of weakness, rapid fatigue and anorexia, had relief of all symptoms after splenic irradiation

### Definitions

For convenience, certain terms employed in the specification, examples, and appended claims are collected here.

The articles “a” and “an” are used herein to refer to one or to more than one (i.e. to at least one) of the grammatical object of the article. By way of example, “an element” means one element or more than one element.

The term “radionuclide” refers to a radioactive isotope or element.

The term “biodistribution” refers to the location of the given particle or particles in a biological entity.

The term “microsphere” refers to an object that is substantially spherical in shape and has a diameter less than 1 millimeter.

The term “glass” refers to a hard, brittle, non-crystalline, inorganic substance, which is usually transparent; glasses are often made by fusing silicates with soda, as described by Webster's New World Dictionary. Ed. Guralnik, D B 1984.

The phrase “time of use” refers to the period during which a microsphere is implanted in a patient or subject.

For purposes of this invention, the chemical elements are identified in accordance with the Periodic Table of the Elements, CAS version, Handbook of Chemistry and Physics, 67th Ed., 1986-87, inside cover.

### Microspheres of the Invention

One aspect of the present invention relates to a microsphere, comprising a material selected from the group consisting of glass, polymer and resin; a first radioisotope that emits a therapeutic β-particle; and a second radioisotope that emits a diagnostic γ-ray; wherein the atomic number of the first radioisotope is not the same as the atomic number of the second radioisotope.

In certain embodiments, the present invention relates to the aforementioned microsphere and the attendant definitions, wherein the ratio of the radioactivity of the second radioisotope to the first radioisotope is in the range from about 1:10 to about 1:10⁷ at the time of use.

In certain embodiments, the present invention relates to the aforementioned microsphere and the attendant definitions, wherein the ratio of the radioactivity of the second radioisotope to the first radioisotope is in the range from about 1:10² to 1:10⁶ at the time of use.

In certain embodiments, the present invention relates to the aforementioned microsphere and the attendant definitions, wherein the ratio of the radioactivity of the second radioisotope to the first radioisotope is in the range from about 1:10⁴ to 1:10⁵ at the time of use.

In certain embodiments, the present invention relates to the aforementioned microsphere and the attendant definitions, wherein said material is selected from the group consisting of glass and polymer.

In certain embodiments, the present invention relates to the aforementioned microsphere and the attendant definitions, wherein said material is glass.

In certain embodiments, the present invention relates to the aforementioned microsphere and the attendant definitions, wherein the diameter of said microsphere is in the range from about 5-75 micrometers.

In certain embodiments, the present invention relates to the aforementioned microsphere and the attendant definitions, wherein the diameter of said microsphere is in the range from about 5-500 micrometers.

In certain embodiments, the present invention relates to the aforementioned microsphere and the attendant definitions, wherein the diameter of said microsphere is in the range from about 10-100 micrometers.

In certain embodiments, the present invention relates to the aforementioned microsphere and the attendant definitions, wherein the diameter of said microsphere is in the range from about 20-50 micrometers.

In certain embodiments, the present invention relates to the aforementioned microsphere and the attendant definitions, wherein said microsphere is solid, hollow, or comprises a plurality of hollow cells.

In certain embodiments, the present invention relates to the aforementioned microsphere and the attendant definitions, wherein said microsphere is solid or hollow.

In certain embodiments, the present invention relates to the aforementioned microsphere and the attendant definitions, wherein said microsphere is solid.

In certain embodiments, the present invention relates to the aforementioned microsphere and the attendant definitions, wherein the density of said microsphere is in the range from about 1.0-4.0 grams/cubic centimeter.

In certain embodiments, the present invention relates to the aforementioned microsphere and the attendant definitions, wherein the density of said microsphere is in the range from about 1.0-3.0 grams/cubic centimeter.

In certain embodiments, the present invention relates to the aforementioned microsphere and the attendant definitions, wherein the density of said microsphere is in the range from about 1.0-2.0 grams/cubic centimeter.

In certain embodiments, the present invention relates to the aforementioned microsphere and the attendant definitions, wherein said first radioisotope is not leached from said microsphere to an extent greater than about 3%; wherein said second radioisotope is not leached from said microsphere to an extent greater than about 3%.

In certain embodiments, the present invention relates to the aforementioned microsphere and the attendant definitions, wherein said first radioisotope is not leached from said microsphere to an extent greater than about 1%; wherein said second radioisotope is not leached from said microsphere to an extent greater than about 1%.

In certain embodiments, the present invention relates to the aforementioned microsphere and the attendant definitions, wherein said first radioisotope is ⁹⁰Y or ³²P.

In certain embodiments, the present invention relates to the aforementioned microsphere and the attendant definitions, wherein said first radioisotope is ⁹⁰Y.

In certain embodiments, the present invention relates to the aforementioned microsphere and the attendant definitions, wherein said second radioisotope is ¹⁹⁸Au.

In certain embodiments, the present invention relates to the aforementioned microsphere and the attendant definitions, wherein said first radioisotope is ⁹⁰Y or ³²P; and said second radioisotope is ¹⁹⁸Au.

In certain embodiments, the present invention relates to the aforementioned microsphere and the attendant definitions, wherein said first radioisotope is ⁹⁰Y; and said second radioisotope is ¹⁹⁸Au.

### Methods of the Invention

The present invention also relates to a method of preparing a radioactive microsphere, comprising the steps of:

combining a non-radioactive precursor of a first radioisotope, a non-radioactive precursor of a second radioisotope, and a material selected from the group consisting of glass, polymer, and resin, to form a mixture; wherein the atomic number of the first radioisotope is not the same as the atomic number of the second radioisotope;

fabricating a microsphere from said mixture; and

bombarding said microsphere with neutrons.

In certain embodiments, the present invention relates to the aforementioned method and the attendant definitions, wherein said material is glass; said non-radioactive precursor of a first radioisotope is Y; and said non-radioactive precursor of a second radioisotope is Au.

In certain embodiments, the present invention relates to the aforementioned method and the attendant definitions, wherein the ratio of the radioactivity of the second radioisotope to the first radioisotope is in the range from about 1:10 to about 1:10⁷.

In certain embodiments, the present invention relates to the aforementioned method and the attendant definitions, wherein the ratio of the radioactivity of the second radioisotope to the first radioisotope is in the range from about 1:10² to 1:10⁶.

In certain embodiments, the present invention relates to the aforementioned method and the attendant definitions, wherein the ratio of the radioactivity of the second radioisotope to the first radioisotope is in the range from about 1:10⁴ to 1:10⁵.

Another aspect of the present invention relates to a method of treating a mammal suffering from a medical condition, comprising the step of:

administering to said mammal a therapeutically effective amount of radioactive microspheres each comprising a material selected from the group consisting of glass, polymer, and resin; a first radioisotope that emits a therapeutic β-particle; and a second radioisotope that emits a diagnostic γ-ray; wherein the atomic number of the first radioisotope is not the same as the atomic number of the second radioisotope.

In certain embodiments, the present invention relates to the aforementioned method and the attendant definitions, wherein the ratio of the radioactivity of the second radioisotope to the first radioisotope is in the range from about 1:10 to about 1:10⁷ at the time of use.

In certain embodiments, the present invention relates to the aforementioned method and the attendant definitions, wherein the ratio of the radioactivity of the second radioisotope to the first radioisotope is in the range from about 1:10² to 1:10⁶ at the time of use.

In certain embodiments, the present invention relates to the aforementioned method and the attendant definitions, wherein the ratio of the radioactivity of the second radioisotope to the first radioisotope is in the range from about 1:10⁴ to 1:10⁵ at the time of use.

In certain embodiments, the present invention relates to the aforementioned method and the attendant definitions, wherein said material is glass.

In certain embodiments, the present invention relates to the aforementioned method and the attendant definitions, wherein said first radioisotope is ⁹⁰Y or ³²P,

In certain embodiments, the present invention relates to the aforementioned method and the attendant definitions, wherein said first radioisotope is ⁹⁰Y.

In certain embodiments, the present invention relates to the aforementioned method and the attendant definitions, wherein said second radioisotope is ¹⁹⁸Au.

In certain embodiments, the present invention relates to the aforementioned method and the attendant definitions, wherein said material is glass; said first radioisotope is ⁹⁰Y or ³²P; and said second radioisotope is ¹⁹⁸Au.

In certain embodiments, the present invention relates to the aforementioned method and the attendant definitions, wherein said material is glass; said first radioisotope is ⁹⁰Y; and said second radioisotope is ¹⁹⁸Au.

In certain embodiments, the present invention relates to the aforementioned method and the attendant definitions, wherein said microspheres are administered using a catheter or a syringe.

In certain embodiments, the present invention relates to the aforementioned method and the attendant definitions, wherein said microspheres are administered by a catheter.

### Exemplification

The invention now being generally described, it will be more readily understood by reference to the following examples, which are included merely for purposes of illustration of certain aspects and embodiments of the present invention, and are not intended to limit the invention.

### Example 1

### Calculation of Optimal Amount of ¹⁹⁷Au Required for ⁸⁹Y-containing Glass Microsphere

Experimental Design

In the present case, we wish to label radioactive ⁹⁰Y-containing glass microspheres with a sufficient amount of ¹⁹⁸Au so that the microspheres are detectable by a gamma camera. For the purpose of the example, the microspheres are of the same composition as commercial Theraspheres (40% Y₂O₃ by weight, or 31% Y), except for the presence of the gold compound. The process is to be carried out by neutron activating glass microspheres that contain the stable isotopes Y⁸⁹ and Au¹⁹⁷. In this example, we wish to compute the amount of initial Au¹⁹⁷ that is required, if the desired radioactivity at the time of removal of the sample from the neutron flux is to be 100 mCi of Y⁹⁰ and 1 μCi of Au¹⁹⁸ Per 50 mg of glass microspheres. The neutron capture cross-sections for Y⁸⁹ and Au¹⁹⁷ are 1.3 barns and 98.8 barns, respectively, and the decay constants for Y⁹⁰ and Au¹⁹⁸ are 3.01×10⁻⁶ s⁻¹ and 2.98×10⁻⁶s⁻¹, respectively. Supposing that the neutron flux is 1×10¹⁴ cm⁻²s⁻¹, the neutron capture constants are computed from Eq 4 to be 1.3×10⁻¹⁰s⁻¹ for Y⁸⁹ and 9.88×10⁻⁹s⁻¹ for Au¹⁹⁷. For both elements, the neutron capture constant is, to a good approximation, negligible compared to the decay constant. Under this circumstance, and as long as the neutron activation time (t) is less than about 5×10⁶ s, Eq 6 is approximated by Eq 8, to within about 5%:

k_{N}=ø_{χ} (Eq 4)

$$\begin{matrix}\frac{ⅆ {A}^{*}}{ⅆ t} = - {k}_{D} {A}^{*} & ( Eq 5 ) \\ \frac{- ⅆ {A}^{*}}{ⅆ t} = \frac{{k}_{D} {k}_{N} {A}_{0} ( {ⅇ}^{- {k}_{N} t} - {ⅇ}^{- {k}_{D} t} )}{{k}_{D} - {k}_{N}} & ( Eq 6 ) \\ \frac{- ⅆ {A}^{*}}{ⅆ t} \approx {k}_{N} {A}_{0} ( 1 - {ⅇ}^{- {k}_{D} t} ) & ( Eq 8 )\end{matrix}$$

Using Eq 8, we calculate that activating the 50 mg of glass microspheres (0.174 mmol of Y) to a radioactivity of 100 mCi will require about 1.05×10⁵ s. Substituting values for the Au and Y isotopes into Eq 8 and forming ratios, and noting that the decay constants are nearly identical, we arrive at the final equation which expresses the ratio of radioactivity to the ratio of the initial amounts of stable isotopes:

$$\frac{\frac{ⅆ ( A {u}^{198} )}{ⅆ t}}{\frac{ⅆ ( {Y}^{90} )}{ⅆ t}} = \frac{{χ}_{A {u}^{197}} A {u}_{0}^{197}}{{χ}_{{Y}^{89}} {Y}_{0}^{89}} = \frac{76 A {u}_{0}^{197}}{{Y}_{0}^{89}} \qquad ( Eq 9 )$$

For the present example, substituting the desired radioactivity values in Eq 9 gives the starting mole ratio of gold to yttrium as:

$$\frac{A {u}_{0}^{197}}{{Y}_{0}^{89}} = 1.32 \times {10}^{- 7} ( mole ratio ) = 2.92 \times {10}^{- 7} ( mass ratio )$$

In the glass composition, which is 31% Y by weight, the necessary amount of gold is finally computed to be 91 Parts per billion, by weight.

### Example 2

In this example, we assume a glass composition containing 13% Y₂O₃ by weight (or 10% Y), and desire radioactivities of 100 mCi for Y⁹⁰ and 10 μCi for Au¹⁹⁸ Per 50 mg of glass microspheres. The other quantities are as for Example 1. A similar computation shows that the glass should contain 291 Ppb of gold, and requires a neutron activation time of 6.10×10⁵ s. Similar calculations may be performed for other proportions of these elements or combinations of other elements in any proportions.

### Example 3

Glass Bead Preparation

The procedures for preparing glass microspheres has been reported previously. See U.S. Pat. No. 5,302,369. In these preparations, glass of varying compositions of Si, Al, K, Mg, Al, Pb, and P₂O₅ has been prepared using reagent grade chemicals. Batches yielding 50 grams of glass were melted in platinum crucibles in an electric furnace at the approximate temperatures. A typical melting cycle required three hours for batch additions at 1000° C. and three to four hours to refine the melt at the approximate melting temperature. The crucible containing the melt was quenched in 25° C. water, after which the resultant glass frit was broken from the crucible and ground to −100 mesh. The −100 mesh glass powder was then slowly fed by a vibrating spatula into an oxygen/propane flame where surface tension pulled the molten particles into spheres. The flow rates of oxygen and propane were adjusted for each glass composition so as to yield the highest fraction of spherical particles. After spheroidizing, the microspheres were wet screened with deionized water, rinsed in acetone and dried.

### Incorporation by Reference

All of the patents and publications cited herein are hereby incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

We claim:

1. A microsphere, comprising a material selected from the group consisting of glass, polymer and resin; a first radioisotope that emits a therapeutic β-particle; and a second radioisotope that emits a diagnostic γ-ray; wherein the atomic number of the first radioisotope is not the same as the atomic number of the second radioisotope.

2. The microsphere of claim 1, wherein the ratio of the radioactivity of the second radioisotope to the first radioisotope is in the range from about 1:10 to about 1:10⁷ at the time of use.

3. The microsphere of claim 1, wherein the ratio of the radioactivity of the second radioisotope to the first radioisotope is in the range from about 1:10² to 1:10⁶ at the time of use.

4. The microsphere of claim 1, wherein the ratio of the radioactivity of the second radioisotope to the first radioisotope is in the range from about 1:10⁴ to 1:10⁵ at the time of use.

5. The microsphere of claim 1, wherein said material is selected from the group consisting of glass and polymer.

6. The microsphere of claim 1, wherein said material is glass.

7. The microsphere of claim 1, wherein the diameter of said microsphere is in the range from about 5-75 micrometers.

8. The microsphere of claim 1, wherein the diameter of said microsphere is in the range from about 5-500 micrometers.

9. The microsphere of claim 1, wherein the diameter of said microsphere is in the range from about 10-100 micrometers.

10. The microsphere of claim 1, wherein the diameter of said microsphere is in the range from about 20-50 micrometers.

11. The microsphere of claim 1, wherein said microsphere is solid, hollow, or comprises a plurality of hollow cells.

12. The microsphere of claim 1, wherein said microsphere is solid or hollow.

13. The microsphere of claim 1, wherein said microsphere is solid.

14. The microsphere of claim 1, wherein the density of said microsphere is in the range from about 1.0-4.0 grams/cubic centimeter.

15. The microsphere of claim 1, wherein the density of said microsphere is in the range from about 1.0-3.0 grams/cubic centimeter.

16. The microsphere of claim 1, wherein the density of said microsphere is in the range from about 1.0-2.0 grams/cubic centimeter.

17. The microsphere of claim 1, wherein said first radioisotope is not leached from said microsphere to an extent greater than about 3%; wherein said second radioisotope is not leached from said microsphere to an extent greater than about 3%.

18. The microsphere of claim 1, wherein said first radioisotope is not leached from said microsphere to an extent greater than about 1%; wherein said second radioisotope is not leached from said microsphere to an extent greater than about 1%.

19. The microsphere of claim 1, wherein said first radioisotope is ⁹⁰Y ³²P.

20. The microsphere of claim 1, wherein said first radioisotope is ⁹⁰Y.

21. The microsphere of claim 1, wherein said second radioisotope is ¹⁹⁸Au.

22. The microsphere of claim 1, wherein said first radioisotope is ⁹⁰Y or ³²P; and said second radioisotope is ¹⁹⁸Au.

23. The microsphere of claim 1, wherein said first radioisotope is ⁹⁰Y; and said second radioisotope is ¹⁹⁸Au.

24. A method of preparing a radioactive microsphere, comprising the steps of:
combining a non-radioactive precursor of a first radioisotope, a non-radioactive precursor of a second radioisotope, and a material selected from the group consisting of glass, polymer, and resin, to form a mixture; wherein the atomic number of the first radioisotope is not the same as the atomic number of the second radioisotope;
fabricating a microsphere from said mixture; and
bombarding said microsphere with neutrons.

25. The method of claim 24, wherein said material is glass; said non-radioactive precursor of a first radioisotope is Y; and said non-radioactive precursor of a second radioisotope is Au.

26. The method of claim 24, wherein the ratio of the radioactivity of the second radioisotope to the first radioisotope is in the range from about 1:10 to about 1:10⁷.

27. The method of claim 24, wherein the ratio of the radioactivity of the second radioisotope to the first radioisotope is in the range from about 1:10² to 1:10⁶.

28. The method of claim 24, wherein the ratio of the radioactivity of the second radioisotope to the first radioisotope is in the range from about 1:10⁴ to 1:10⁵.

29. A method for treating a mammal suffering from a medical condition, comprising the step of:
administering to said mammal a therapeutically effective amount of radioactive microspheres each comprising a material selected from the group consisting of glass, polymer, and resin; a first radioisotope that emits a therapeutic β-particle; and a second radioisotope that emits a diagnostic γ-ray; wherein the atomic number of the first radioisotope is not the same as the atomic number of the second radioisotope.

30. The method of claim 29, wherein the ratio of the radioactivity of the second radioisotope to the first radioisotope is in the range from about 1:10 to about 1:10⁷ at the time of use.

31. The method of claim 29, wherein the ratio of the radioactivity of the second radioisotope to the first radioisotope is in the range from about 1:10² to 1:10⁶ at the time of use.

32. The method of claim 29, wherein the ratio of the radioactivity of the second radioisotope to the first radioisotope is in the range from about 1:10⁴ to 1:10⁵ at the time of use.

33. The method of claim 29, wherein said material is glass.

34. The method of claim 29, wherein said first radioisotope is ⁹⁰Y or ³²P.

35. The method of claim 29, wherein said first radioisotope is ⁹⁰Y.

36. The method of claim 29, wherein said second radioisotope is ¹⁹⁸Au.

37. The method of claim 29, wherein said material is glass; said first radioisotope is ⁹⁰Y or ³²P; and said second radioisotope is ¹⁹⁸Au.

38. The method of claim 29, wherein said material is glass; said first radioisotope is ⁹⁰Y; and
said second radioisotope is ¹⁹⁸Au.

39. The method of claim 29, wherein said microspheres are administered using a catheter or a syringe.

40. The method of claim 29, wherein said microspheres are administered by a catheter.
